(19) United States
(12) Patent Application Publication
(10) Pub. No.: US 2025/0312098 A1
(43) Pub. Date: Oct. 9, 2025

(54) DEVICES, METHODS, AND SYSTEMS FOR IMPROVED PLANNING AND GUIDANCE IN LASER FENESTRATION APPLICATIONS

(71) Applicant: KONINKLIJKE PHILIPS N.V., EINDHOVEN (NL)

(72) Inventors: Vipul Shrihari PAI RAIKAR, SOMERVILLE, MA (US); Alyssa TORJESEN, EXETER, NH (US); Brian C. LEE, CAMBRIDGE, MA (US); Manfred MÜLLER, EINDHOVEN (NL); Rene Leonardus Jacobus Marie UBACHS, EINDHOVEN (NL); Torre Michelle BYDLON, MELROSE, MA (US); Marco LAI, EINDHOVEN (NL)

(21) Appl. No.: 18/864,998

(22) PCT Filed: May 17, 2023
(86) PCT No.: PCT/EP2023/063212

§ 371 Date: Nov. 12, 2024

Related U.S. Application Data
(60) Provisional application No. 63/344,880, filed on May 23, 2022.

(51) Int. Cl. A61B 18/24; A61B 18/00; A61B 34/10; A61B 90/00

(52) U.S. Cl. CPC A61B 18/24; A61B 34/10; A61B 90/37; A61B 2018/00166; A61B 2034/107

(57) ABSTRACT

A system is provided for fenestration within a body lumen. The system includes an intraluminal device and a processor. The processor is configured to, from an imaging system, obtain a planning image including the lumen and a branch lumen extending from the lumen, and, in the planning image: identify a treatment device; identify a centerline of the branch lumen extending from the branch lumen to a desired puncture point on the treatment device; and identify a desired trajectory of the intraluminal device relative to the puncture point. The processor is also configured to, from a second imaging system, obtain a live procedural image including the lumen and the branch lumen; and in the live procedural image, identify the treatment device, the centerline of the branch lumen, the desired puncture point, the desired trajectory of the intraluminal device, and the actual trajectory of the intraluminal device.

### TECHNICAL FIELD

The subject matter described herein relates to a devices, systems, and methods for intraluminal therapeutic fenestration (e.g., puncture) of vascular stent grafts. This technology has particular but not exclusive utility for improving the speed and quality of aortic aneurism repairs.

### INTRODUCTION

The aorta is the main artery that transports blood from the heart to the rest of the body. Blood leaves the heart through the aortic valve, then travels through the aorta, from which branching arteries deliver blood to the limbs and organs. An aortic aneurysm is a balloon-like bulge in the aorta, which can occur as a result of disease or injury. Aortic aneurysms can dissect or rupture, e.g., the internal pressure can split the layers of the artery wall, allowing blood to leak in between them or into the chest or abdominal cavity.

Complex endovascular aneurysm repair (EVAR) techniques intended to exclude aortic aneurysms, while preserving blood flow into critical vessels that are anatomically included in the sealing zones, are ideally performed using bespoke custom-made devices (CMDs). This ideal is often compromised by clinical urgency (e.g., emergency surgery following an accident), which has spawned a range of alternative technical options including complex “off-the-shelf” branched devices, chimney/periscope/snorkel EVAR (CHIMPS), and physician-modified endografts (PMEGs). The latter modifications can be done on the bench prior to implantation or in-vivo. In-situ laser fenestration (LfEVAR, e.g., puncturing the stent graft from inside, using an intravascular laser catheter) is an example of the latter. In-situ laser fenestration utilizes laser light energy to produce deliberate holes in the graft fabric of the main device after its deployment, which can be dilated and used as fenestrations for urgent fenestrated EVAR and similar procedures.

The principle is to generate a small starting hole (for dilatation and stenting) in the fabric of a deployed endograft using a contact laser fiber exactly sited over the center of the target vessel ostium. Depending on the anatomy, there are two possible approaches to perform in situ fenestrations: anterograde (e.g., from the aorta to the target vessel) and retrograde (e.g., from the target vessel to the aorta). Each of these approaches has its own technical challenges and ancillary tools.

The retrograde approach may utilize vascular access downstream from the target artery ostium. The laser probe is introduced in a retrograde fashion and once in contact with the graft, its tip is positioned square-on or flush to the endograft fabric in order to deliver the maximum amount of energy to the smallest surface area in an effort to create a circular, nonelliptical hole. The anterograde approach may be more challenging. For example, precise positioning of the laser fiber and a high degree of stability are desirable, but difficult to achieve with existing approaches. In some instances, steerable sheaths may be used to guide the positioning of the laser fiber. The radiopaque tip of the steerable sheath may be positioned at the level of the ostium of the target artery and confirmed via fluoroscopy (e.g., two orthogonal fluoroscopy views).

Once the laser fiber is in contact with the polyester fabric of the stent graft, and correctly positioned and stabilized by the steerable sheath, the laser generator is activated (e.g., for 3 seconds). A guidewire (e.g., a 0.014-inch diameter guidewire) can then be inserted via the monorail laser fiber and advanced through the fenestration into the target vessel. Successive balloon dilatations of the graft fenestration may be required to progressively enlarge it. A 2- or 2.5-mm-diameter cutting balloon can be used for visceral arteries and a 6-mm cutting balloon for the supra-aortic trunk for the first dilatation and follow with a semi-compliant 4-mm-diameter coronary artery balloon. The 0.014-inch wire may be exchanged for a 0.035-inch Rosen wire, and a 6-F sheath advanced into the target vessel to facilitate positioning and deployment of the bridging (covered) balloon-expandable stent, which may be inflated with 3 to 4 mm protruding into the aortic lumen for final flaring (e.g., using a 9- or 10-mm-diameter balloon).

In situ laser fenestrations are a useful adjunct for EVAR when the proximal landing zone includes the origin of the visceral (abdominal) or the great (arch) vessels. Use of CMDs remains the first choice when it is safe to delay treatment. Laser fenestration is recommended in urgent situations in fragile patients not thought to be good candidates for open surgical repair. Laser fenestration is also useful in the treatment of chronic aortic dissections. The laser fiber can be used to fenestrate the dissection flap to navigate from one lumen to the other. This can be a quick maneuver, and may be less aggressive or risky than the alternative solutions that employ transseptal needles or a rigid guidewire tip.

The reported median time to perform four fenestrations is <1 hour. A potential advantage of in situ fenestration over the PMEG technique is rapid exclusion of the aneurysm, which can be advantageous when there is rupture. Laser fenestration may for example be indicated for treatment of tender or ruptured juxtarenal aneurysms, and for symptomatic patients with type Ia endoleaks complicating previous EVAR. Occasionally, it has also been used to treat thoracoabdominal aneurysms, aortic arch aneurysms, and preserved large renal polar arteries using laser fenestrations. Additionally, the principles have been used to convert a previously implanted aorto-uni-iliac stent graft into an aorto-biiliac stent graft.

Once the origin of a side-branching vessel has been covered by the endograft fabric, it may be difficult to locate by angiography. Pre-stenting of the target branch arteries and/or image fusion technology can be used to overcome this issue. For pre-stenting a target branch vessel, balloon-expandable stents may be positioned within the target branch vessels at the beginning of the procedure, prior to inserting the aortic endograft. These stents may be deployed with their proximal extent at the vessel ostium. If deployed too far into in the target branch artery, the stent may not be useful in the precise location of the origin of the target branch vessel and subsequent positioning of the tip of the steerable sheath; if deployed too medially with protrusion into the aortic lumen, there are risks of crushing the target branch vessel stent and disturbing the main aortic device seal.

There can be a risk of dissection during pre-stenting, as well as a risk of the 0.014-inch guidewire colliding with the stent struts and a risk of stent migration when advancing the introducer sheath. The laser fiber diameter for anterograde visceral artery laser fenestrations may be 0.9 mm, while a 2.3-mm-diameter fiber may be used for retrograde fenestration of the supra-aortic trunks, though other size devices may be used for both anterograde and retrograde procedures. The precise and stable positioning of the laser fiber may be challenging. The radio-opaque tip of the steerable sheath (or the laser catheter in general) may be positioned at the level of the ostium of the target artery and confirmed, for example, on two orthogonal fluoroscopy views. Furthermore, the laser catheter cannot bend beyond a certain radius of curvature and still function properly.

Type II endoleaks after endovascular abdominal aortic aneurysm repair can also occur (e.g., as a result of retrograde flow from arterial aortic side branches refilling the aneurysm sac). Type II endoleaks are complex vascular structures that may contain an endoleak cavity, or nidus, with several feeding and draining vessels, similar to an arteriovenous malformation. Some type II endoleaks are transient and either resolve spontaneously within a few months or remain benign. However, persistent type II endoleaks can be associated with sac expansion and may, therefore, require secondary interventions to avoid rupture. Currently, it may be difficult to judge the ideal amount of glue for embolization that should be used for sealing the endoleak.

A C-arm is a type of x-ray imaging system that can be rotated around several different axes. During a procedure it may be desirable for the C-arm angle to be set such that the scanner images the target vessels with minimal obstruction by other body structures. However, it can be difficult to know ahead of time which C-arm angle or angles will optimize the view of the target vessel(s), which can interfere with registration and/or landmark identification between the planning CT and live fluoroscopy images.

Thus, it is to be appreciated that existing intravascular stent graft fenestration techniques can have numerous drawbacks, including difficulty in identifying the correct location to puncture the stent graft material, difficulty in aligning the laser catheter on the correct spot, difficulty in forming a circular (as opposed to elliptical or irregular shaped) opening, difficulty in aligning the laser catheter without over-bending, etc. Accordingly, a need exists for improved stent graft fenestration devices, systems, and methods that address one or more of the forgoing and/or other concerns.

The information included in this Introduction section of the specification, including any references cited herein and any description or discussion thereof, is included for technical reference purposes only and is not to be regarded as subject matter by which the scope of the disclosure is to be bound.

### SUMMARY

Disclosed are devices, systems, and methods for improving navigation of catheters and guidewires during a laser fEVAR technique, endoleak repair, or other intravascular fenestration procedures. In particular, the improved navigation may be provided by: pre-planning of the puncturing point on the stent graft in 3D; defining the catheter laser path in 3D during the surgical planning; adjusting the pre-plan after that the main stent graft is positioned, in order to avoid the stent struts during laser perforation and to improve blood flow; and image segmenting on the fluoroscopy of the laser catheter to assess whether the catheter follows the 3D planned trajectory. In the case of a laser fEVAR procedure, improved navigation and outcomes may be provided by: Enlarging the opening via a cutting balloon or other tool to a size that correctly matches the diameter of the artery branch, displayed in the surgical planning. In the case of an endoleak repair, improved navigation and outcomes may be provided by: planning and marking the region of the branch artery that is to be plugged with glue; computing the volume of glue to inject; and once the glue catheter is in position at the distal end of the glue injection region, injecting the computed amount of glue while the glue catheter is withdrawn.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a system for intraluminal fenestration within a body lumen. The system includes a first intraluminal device including a flexible elongate member; and a processor in communication with the first intraluminal device, where the processor is configured to: obtain a planning image from a first imaging system, the planning image including the body lumen and a branch lumen extending from the body lumen; in the planning image: identify a profile of a treatment device; identify a centerline of the branch lumen, the centerline of the branch lumen extending from the branch lumen to a desired puncture point at a boundary of the profile of the treatment device; and identify a desired trajectory of the first intraluminal device relative to the desired puncture point. The processor is further configured to obtain a live procedural image from a second imaging system, the live procedure image including the body lumen and the branch lumen extending from the body lumen; and in the live procedural image, identify the profile of the treatment device, the centerline of the branch lumen, the desired puncture point, the desired trajectory of the first intraluminal device, and an actual trajectory of the first intraluminal device. Other examples of this aspect include corresponding computer systems, apparatus, and computer programs recorded on one or more computer storage devices, each configured to perform the actions of the methods.

Implementations may include one or more of the following features. In some aspects, the processor is further configured to: when the first intraluminal device is within a desired proximity and angle of the desired puncture point, activate a functional portion of the first intraluminal device to puncture the treatment device. In some aspects, the processor is further configured to: in the planning image, identify lumen walls of the branch lumen, extending from the branch lumen to the profile of the treatment device; and in the live procedural image, identify the lumen walls of the branch lumen, extending from the branch lumen to the profile of the treatment device. In some aspects, the processor is further configured to: after the treatment device is placed within the body lumen, based on a position of the treatment device within the live procedural image, adjust the identified desired puncture point and the identified desired trajectory of the first intraluminal device. In some aspects, the processor is further configured to: display, on the live procedural image, an indication of a difference between the desired trajectory of the first intraluminal device and the actual trajectory of the first intraluminal device. In some aspects, the guide catheter or guidewire is configured to affect the actual trajectory of the first intraluminal device. In some aspects, the guidewire is a fiber optic real shape (FORS) guidewire, and the processor is further configured to, as the FORS guidewire moves through the body lumen, identify, on the live procedural image, a 3D trajectory of the FORS guidewire. In some aspects, the processor is further configured to: as a second intraluminal device moves through the body lumen, identify, on the live procedural image, an actual trajectory of the second intraluminal device. In some aspects, the guide catheter or guidewire is configured to affect the actual trajectory of the second intraluminal device. In some aspects, the second intraluminal device is a cutting device configured to increase a diameter of a puncture in the treatment device. In some aspects, the second intraluminal device is an intravascular imaging device. In some aspects, the second intraluminal device is an injection catheter configured to inject a therapeutic material. In some aspects, the processor is further configured to: in the planning image, identify an injection volume within the branch lumen, where the injection volume is to be injected with the therapeutic material; and in the live procedural image, identify the injection volume within the branch lumen. In some aspects, the profile of the treatment device is a desired profile for a treatment device to be implanted or an actual profile of a treatment device already implanted. The first intraluminal device is a laser catheter device. In some aspects, the processor is further configured to identify the desired trajectory of the first intraluminal device such that a bend radius of the first intraluminal device is not smaller than a specified bend radius. In some aspects, the processor is further configured to identify the desired trajectory of the first intraluminal device such that a functional portion of the first intraluminal device approaches the desired puncture point within a desired range of angles. Implementations of the described techniques may include hardware, a method or process, or computer software on a computer-accessible medium.

One general aspect includes a method for intraluminal fenestration within a body lumen. The method includes, with a processor in communication with a first intraluminal device including a flexible elongate member: obtaining a planning image from a first imaging system, the planning image including the body lumen and a branch lumen extending from the body lumen; in the planning image, identifying a profile of a treatment device; in the planning image, identifying a centerline of the branch lumen, the centerline of the branch lumen extending from the branch lumen to a desired puncture point at a boundary of the profile of the treatment device; in the planning image, identifying a desired trajectory of the first intraluminal device relative to the desired puncture point; obtaining a live procedural image from a second imaging system, the live procedure image including the body lumen and the branch lumen extending from the body lumen; and in the live procedural image, identifying the profile of the treatment device, the centerline of the branch lumen, the desired puncture point, the desired trajectory of the first intraluminal device, and an actual trajectory of the first intraluminal device. Other embodiments of this aspect include corresponding computer systems, apparatus, and computer programs recorded on one or more computer storage devices, each configured to perform the actions of the methods.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the laser fenestration systems, devices, and methods as defined in the claims, is provided in the following written description of various aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1A is a diagrammatic, schematic representation of at least a portion of an intraluminal stent graft fenestration system, in accordance with aspects of the present disclosure.

FIG. 1B is a diagrammatic, schematic representation of at least a portion of an intraluminal stent graft fenestration system, in accordance with aspects of the present disclosure.

FIG. 2 is a diagrammatic, schematic representation of at least a portion of an intraluminal stent graft fenestration system, in accordance with aspects of the present disclosure.

FIG. 3 is a diagrammatic, schematic representation of at least a portion of an intraluminal stent graft fenestration system, in accordance with aspects of the present disclosure.

FIG. 4 is a diagrammatic, schematic representation of at least a portion of an intraluminal stent graft fenestration system, in accordance with aspects of the present disclosure.

FIG. 5 is a diagrammatic, schematic representation of at least a portion of an intraluminal stent graft fenestration system, in accordance with aspects of the present disclosure.

FIG. 6 is a diagrammatic, schematic representation of at least a portion of an intraluminal stent graft fenestration system, in accordance with aspects of the present disclosure.

FIG. 7 is a diagrammatic, schematic representation of at least a portion of an intraluminal stent graft fenestration system, in accordance with aspects of the present disclosure.

FIG. 8 is a diagrammatic, schematic representation of at least a portion of an intraluminal stent graft fenestration system, in accordance with aspects of the present disclosure.

FIG. 9 is a diagrammatic, schematic representation of at least a portion of an intraluminal stent graft fenestration system, in accordance with aspects of the present disclosure.

FIG. 10 is a diagrammatic, schematic representation of at least a portion of an intraluminal stent graft fenestration system, in accordance with aspects of the present disclosure.

FIG. 11 is a diagrammatic, schematic representation of a CT planning image showing at least a portion of an intraluminal stent graft fenestration system, in accordance with aspects of the present disclosure.

FIG. 12 is a diagrammatic, schematic representation of a CT planning image showing at least a portion of an intraluminal stent graft fenestration system, in accordance with aspects of the present disclosure.

FIG. 13 is a diagrammatic, schematic representation of a fluoroscopy image showing at least a portion of an intraluminal stent graft fenestration system, in accordance with aspects of the present disclosure.

FIG. 14 is a diagrammatic, schematic representation of a fluoroscopy image showing at least a portion of an intraluminal stent graft fenestration system, in accordance with aspects of the present disclosure.

FIG. 15 is a diagrammatic, schematic representation of a fluoroscopy image and enlarged portion, showing at least a portion of an intraluminal stent graft fenestration system, in accordance with aspects of the present disclosure.

FIG. 16 is a fluoroscopy image of an aortic stent graft, outside of which an endoleak has formed in an aortic side branch artery, in accordance with aspects of the present disclosure.

FIG. 17 is a fluoroscopy image of an aortic stent graft, outside of which an endoleak has formed in an aortic side branch artery, in accordance with aspects of the present disclosure.

FIG. 18 is a fluoroscopy image of an aortic stent graft, outside of which an endoleak has formed in an aortic side branch artery, in accordance with aspects of the present disclosure.

FIG. 19 is a fluoroscopy image of an aortic stent graft, outside of which an endoleak has formed in an aortic side branch artery, in accordance with aspects of the present disclosure.

FIG. 20 is a fluoroscopy image of an aortic stent graft, outside of which an endoleak has formed in an aortic side branch artery, in accordance with aspects of the present disclosure.

FIG. 21 is a diagrammatic, schematic representation of a CT planning image showing at least a portion of an intraluminal stent graft fenestration system, in accordance with aspects of the present disclosure.

FIG. 22 is a diagrammatic, schematic representation of a CT planning image showing at least a portion of an intraluminal stent graft fenestration system, in accordance with aspects of the present disclosure.

FIG. 23 is a diagrammatic, schematic representation of a CT planning image showing at least a portion of an intraluminal stent graft fenestration system, in accordance with aspects of the present disclosure.

FIG. 24 is a diagrammatic, schematic representation of a fluoroscopy image showing at least a portion of an intraluminal stent graft fenestration system, in accordance with aspects of the present disclosure.

FIG. 25 is a diagrammatic, schematic representation of a fluoroscopy image showing at least a portion of an intraluminal stent graft fenestration system, in accordance with aspects of the present disclosure.

FIG. 26A is a diagrammatic, schematic representation of a fluoroscopy image showing at least a portion of an intraluminal stent graft fenestration system, in accordance with aspects of the present disclosure.

FIG. 26B is a diagrammatic, schematic representation of a fluoroscopy image showing at least a portion of an intraluminal stent graft fenestration system, in accordance with aspects of the present disclosure.

FIG. 27A is a diagrammatic, schematic representation of a fluoroscopy image showing at least a portion of an intraluminal stent graft fenestration system, in accordance with aspects of the present disclosure.

FIG. 27B is a diagrammatic, schematic representation of a fluoroscopy image showing at least a portion of an intraluminal stent graft fenestration system, in accordance with aspects of the present disclosure.

FIG. 28 shows a flow diagram of an example stent graft fenestration method, according to aspects of the present disclosure.

FIG. 29 shows a flow diagram of an example stent graft fenestration method, according to aspects of the present disclosure.

FIG. 30 is a schematic diagram of a processor circuit 3050, according to aspects of the present disclosure.

### DETAILED DESCRIPTION

Disclosed are devices, systems, and methods for improving navigation of catheters and guidewires during the laser fEVAR technique, or other intravascular fenestration procedures. In particular, the improved navigation is provided by: pre-planning the puncturing point on the stent graft in 3D; defining the catheter laser path in 3D during the surgical planning; adjusting the pre-plan after that the main stent graft is positioned, in order to avoid the stent struts during laser perforation and to improve blood flow; and image segmenting on the fluoroscopy of the laser catheter to assess whether the catheter follows the 3D planned trajectory. In the case of a laser fEVAR procedure, improved navigation and outcomes may be provided by: enlarging the opening via a cutting balloon or other tool to a size that correctly matches the diameter of the artery branch, displayed in the surgical planning. In the case of an endoleak repair, improved navigation and outcomes may be provided by: planning and marking the region of the branch artery that is to be plugged with glue; computing the volume of glue to inject; and once the glue catheter is in position at the distal end of the glue injection region, injecting the computed amount of glue while the glue catheter is withdrawn.

During the pre-planning, a pre-operative CT angiography (or similar 3D image of the patient) can be used for planning the procedure. The vessels may be segmented from the pre-op CT image and used as a roadmap for the planning. Optimal C-arm angles may then be computed and marked on the pre-op CT, for each artery branch that needs fenestration. A virtual main stent graft is positioned in correspondence with the aortic aneurysm, for planning the locations to puncture with the laser, and also for guiding the placement of the stent graft during surgery. Optimal puncture locations may be determined by evaluating several options using computational modelling. Optimization can be aimed at achieving a certain amount of blood flow into to the side branches.

The artery branch that needs fenestration may then be segmented, and the vessel diameter and the trajectory of the central axis of the vessel can be calculated and marked on the CT image. The targeted vessel trajectory can then be prolonged until it reaches the virtual main stent graft, and its intersection with the virtual stent graft defines the 3D position of the puncturing point of the main stent graft. In some aspects, instead of prolonging the artery trajectory, intersection with the main stent graft can be achieved with another method, for example by projecting orthogonally the artery origin to the axis of the main stent graft.

Definition of the laser catheter path in 3D during the surgical planning:

The optimal 3D path of the laser catheter can be computed, such that the path is contained inside the 3D vessel roadmap segmented from the pre-op CT. Also, the path should prevent the laser catheter from bending beyond a certain bend angle, such that the laser catheter functions properly. The limit angle depends on the specifics of the laser atherectomy device used for laser fenestration. Additionally, the path may be selected to approach the wall of the stent graft as orthogonally as possible (e.g., within a desired range of angles), given the above constraints, such that the resulting fenestration is as circular as possible. The trajectory of the laser catheter may be computed for each artery branch that is going to be fenestrated.

Adjustment of the pre-planning after main stent graft placement:

As the surgical procedure starts, the 3D surgical planning made on the pre-op CT is registered on the patient. A 2D projection of the surgical plan can be overlaid on the 2D fluoroscopy image. The planning can then be used for positioning the main stent graft on the aortic aneurysm. After that the main stent graft is deployed, the C-arm angle for the targeted artery branch is selected and the surgical plan is projected in 2D on the new C-arm angle. On the fluoroscopy, the position of the stent graft and its stent struts are visible.

At this moment, differences between the planned landing site and the actual landing site of the stent are visible and the planning can be adjusted accordingly. For example, the puncture locations and the laser catheter path may be adjusted. In one particular example, the stent struts may interfere with the desired puncturing position of the laser catheter, since a hole cannot readily be opened through the strut material. Therefore, the 3D puncturing position could be shifted, for example above or below the stent struts, in order to reduce interference. Subsequently, the corresponding artery branch trajectory and planned laser catheter path are also shifted or adjusted, and the desired trajectory of the laser catheter can be recalculated based on the updated path to ensure it remains within its operating specifications (bend radius, etc.).

If the stent strut is not exactly on the planned puncturing location of the laser, but still in a nearby area inside the projected diameter of the targeted vessel to be fenestrated, the stent struts may put some pressure on the branch stent graft once it is deployed and cause its closure (or partial closure). In such cases, the planned puncturing position can also be shifted.

Assessment of the laser catheter path on the fluoroscopy:

The laser catheter (e.g. used with a steerable sheath, or guided along a guidewire whose tip is placed against the desired puncture location) is advanced inside the main stent graft until the planned puncturing point is reached. The laser catheter may be guided by the 3D planned trajectory, which is projected in 2D on the fluoroscopy image. The catheter is segmented from the fluoroscopy image (e.g. by image-based segmentation) and matched with the projected planned path in 2D, such that the catheter position can be adjusted, in real time or near-real time, to match the projected plan. In some aspects, the average distance between planned path and segmented catheter position is computed and reported to the user, to assess how well the laser catheter overlays the planned 3D path. The surgeon may be notified by the system (e.g. by a message on the display) if the catheter needs adjustment, or for example in case the angle of the catheter is over the limit bending angle. In other aspects, a range for optimal paths can be displayed, such that the surgeon is guided to position the catheter inside that range. After placing the laser catheter in the correct position, the main stent graft can be punctured.

Enlargement of the hole in the stent graft

Depending on the size of the artery branch, the hole may be enlarged by means of a balloon (e.g. a cutting balloon), in order to match the diameter of the artery branch, projected on the main stent graft in the fluoroscopy image. Afterwards, a guidewire can be advanced through the hole in the main stent graft to reach the artery branch, after which the stent graft branch can be advanced over the guidewire, through the fenestration, and deployed into the artery branch as shown below.

The present disclosure aids substantially in the placement of arterial stent grafts with fenestrated branches, by improving the planning and execution of laser fenestration of the main stent graft. Implemented on a catheterization laboratory workstation in conjunction with imaging systems such as CAT and fluoroscopic X-ray, the laser fenestration system disclosed herein provides practical guidance to the surgeon, both pre-operatively and in real time or near-real time during the stent graft placement procedure, and particularly during fenestration of the main stent graft to enable placement of branch stent grafts. This improved situational awareness transforms a surgical procedure based on subjective judgement and guesswork into one that is guided by evidence-based planning, without the normally routine need to improvise the procedure based on real-time fluoroscopy imaging. This unconventional approach improves the functioning of the systems and processors employed in the stent graft placement procedure, by annotating the fluoroscopy images with the desired catheter trajectory and fenestration geometry.

The laser fenestration system may be implemented, at least in part, as an annotation process viewable on a display, and operated by a control process executing on a processor that accepts user inputs from a keyboard, mouse, or touchscreen interface, and that is in communication with one or more imaging systems. In that regard, the control process performs certain specific operations in response to different inputs or selections made at different times or under different circumstances. Certain structures, functions, and operations of the processor, display, sensors, and user input systems are known in the art, while others are recited herein to enable novel features or aspects of the present disclosure with particularity.

These descriptions are provided for exemplary purposes only, and should not be considered to limit the scope of the disclosure. Certain features may be added, removed, or modified without departing from the spirit of the claimed subject matter.

For the purposes of promoting an understanding of the principles of the present disclosure, reference will now be made to the aspects illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one aspect may be combined with the features, components, and/or steps described with respect to other aspects of the present disclosure. For the sake of brevity, however, the numerous iterations of these combinations will not be described separately.

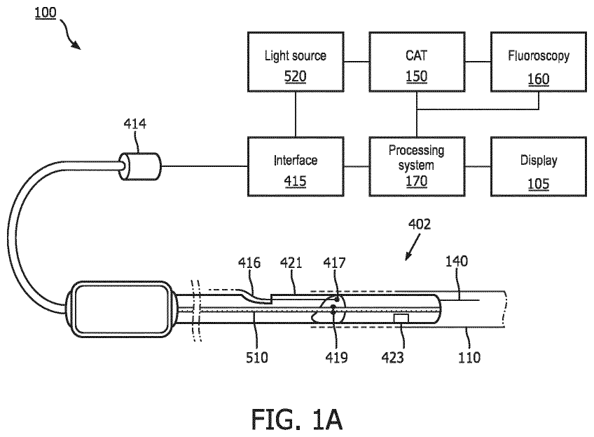

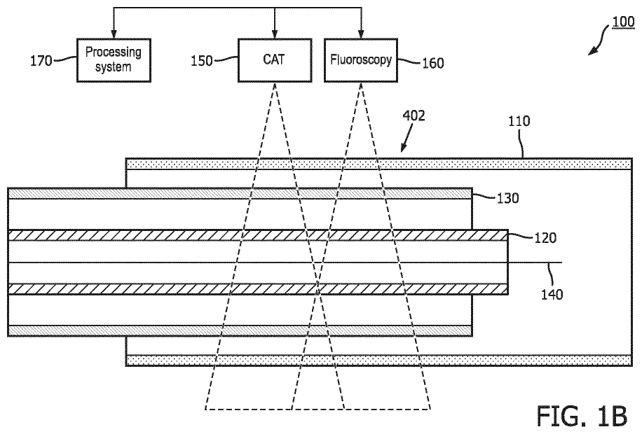

FIG. 1A is a diagrammatic schematic representation of at least a portion of an intraluminal stent graft fenestration system 100, in accordance with aspects of the present disclosure. The intraluminal stent graft fenestration system 100 may include an intraluminal device 402, such as a catheter, guide wire, or guide catheter (see FIG. 1B), an interface 415, a processor, processing system, or controller 170, and a display device 105. At a high level, the intraluminal device 402 emits light longitudinally outward from a distal portion of the intraluminal device and into tissue or implanted therapeutic devices. In this regard, the intraluminal device 402 may be sized, shaped, or otherwise configured to be positioned within a body lumen 110 of a patient. In some aspects, the intraluminal device 402 may include a soft atraumatic tip to track the intraluminal device 402 into the body lumen 110. The body lumen 110 may be a blood vessel, such as an artery or a vein of a patient's vascular system, including cardiac vasculature, peripheral vasculature, neural vasculature, renal vasculature, and/or or any other suitable lumen inside the body. For example, the intraluminal device 402 may be used to direct light into any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the intraluminal device 402 may be used to examine or modify man-made structures such as, but without limitation, heart valves, stents, shunts, filters, grafts, stent grafts, and other devices.

In some aspects, the intraluminal device 402 may include a flexible elongate member 421 that includes an optical fiber 510. In some aspects, the optical fiber 510 is a single optical fiber. In some aspects, the optical fiber 510 includes a plurality of optical fibers. The optical fiber 510 includes a proximal portion and a distal portion. The proximal portion of the optical fiber 510 may be coupled to a light source 520. The proximal portion of the optical fiber 510 may for example be coupled to a light source 520 via the interface 415, as described further below. For example, in some instances the intraluminal device 402 may include and/or be coupled to a connector 414. The connector 414 may be configured to directly or indirectly couple the intraluminal device 402 to the interface 415. The interface 415 may couple the intraluminal device 402 and/or one or more components of the intraluminal device 402 to the light source 520 and/or the processing system 170. In some instances, where the optical fiber 510 is the plurality of optical fibers, each of the optical fibers in the plurality of optical fibers may illuminate several areas of tissue or manmade structure in the body lumen 110 simultaneously. The proximal portion of the optical fiber 510 is configured to receive light from the light source 520. In some instances, where the optical fiber 510 is the plurality of optical fibers, each of the optical fibers in the plurality of optical fibers may be coupled to a same light source 520. In some instances, where the optical fiber 510 is the plurality of optical fibers, one or more of the optical fibers in the plurality of optical fibers may be coupled to different light sources, such as multiple light sources 520. In some instances, when multiple light sources 520 are utilized, the light sources 520 may be configured to emit the same or different light wavelengths.

In some aspects, the light emitted from the light source 520 may be a wavelength in the ultraviolet light spectrum, the visible light spectrum, or the infrared light spectrum. In some aspects, the light emitted from the light source may be a wavelength of between about 300 nanometers (nm) and about 700 nm. In some aspects, the light emitted from the light source may be a UV wavelength of about 300 nm. The distal portion of the optical fiber 510 is configured to be positioned proximate to the tissue or manmade structure, i.e. the region of interest, and emit the light from the light source 520 longitudinally outward from a distal end of the optical fiber 510 and into tissue or manmade structure within the body lumen 110 of the patient. Therefore, in some aspects, the distal portion of the optical fiber 510 may extend longitudinally outward from the end of the flexible elongate member 421. In some aspects, the intraluminal device 402 may be a balloon delivery intraluminal device, injection catheter, intravascular imaging device (e.g., intravascular ultrasound (IVUS), optical coherence tomography (OCT), combinations thereof, etc.), or other device.

The optical fiber 510 may include a core and a cladding positioned around the core. As discussed previously, the distal portion of the optical fiber 510 may be configured to emit the light from the light source 520 longitudinally outward from a distal end of the optical fiber 510 and into tissue or manmade structures within the body lumen 110 of the patient. In some aspects, optical fiber 510 includes one or more scattering elements. The one or more scattering elements may include one or more of an air bubble, a nanosphere, or a microsphere.

In the illustrated example of FIG. 1, the intraluminal device 402 includes a guidewire port 416 and a guidewire lumen 417. In this regard, the intraluminal device 402 may be a rapid-exchange catheter. The guidewire port 416 and the guidewire lumen may allow the intraluminal device 402 to be introduced over a guidewire 140 and into the body lumen 110 of the patient. In some aspects, the intraluminal device 402 includes a guidewire lumen 417 that extends along a majority of a length or the entire length of the intraluminal device 402. In this regard, the intraluminal device 402 may be an over-the-wire catheter. In some instances, the intraluminal device 402 includes one or more optical fiber lumens 419 that receive optical fiber 510 to be positioned within the flexible elongate member 421.

The interface 415 may facilitate communication of signals between the processing system or controller 170, the intraluminal device 402, and/or light source 520. That is, the interface 415 may have appropriate connectors/components for optical, electrical, and/or wireless communication with the processing system or controller 170, the intraluminal device 402, and/or light source 520. In some aspects, the interface 415 and the light source 520 may be one and the same. That is, the intraluminal device 402 may couple directly to the light source 520, which serves as the interface to the processing system 170, controller, or other component of the intraluminal stent graft fenestration system 100.

In some aspects, the intraluminal device 402 may further include a thermal monitoring device 423 that provides an indication of the illumination intensity of the length of the optical fiber 510 where the light is emitted. The processing system or controller 170, which is in communication with the thermal monitoring device 423 and the light source 520 via interface 415, may for example be configured to control one or more attributes of the light source 520 based on feedback received from the thermal monitoring device 423. For example, if the thermal monitoring device 423 detects a temperature that is indicative of the tissue or structure to which the emitted light is exposed may be damaging the tissue or structure, then the processing system or controller 170 may alter one or more attributes of the light source such that the intensity of the light emitted by the light source is decreased. As an alternative example, if the thermal monitoring device 423 detects a temperature that is indicative of the tissue or structure to which the emitted light is exposed may not be generating the desired amount of heat or damage, then the processing system or controller 170 may alter one or more attributes of the light source such that the intensity of the light emitted by the light source is increased. In some aspects, the interface 415 transfers signals including the feedback received from the thermal monitoring device 423 in the intraluminal device 402 to the processing system or controller 170 where the signals may be displayed on the display device 105.

In some aspects, the intraluminal stent graft fenestration system 100 may include a Computer Aided Tomography (CAT or CT) scanner or imaging system 150 and/or a fluoroscopy x-ray system 160, under the control of one or more processors 170. In an example, the CAT scanner 150 or fluoroscopy x-ray system may capture images when commanded by the processing system 170, and/or the CAT scanner 150 or fluoroscopy x-ray system may transmit the images to the processing system 170 for display on the display device 105. In some instances, the processing system 170 may modify the images before displaying them. For example, the processing system 170 may process, enhance, annotate, split, combine, overlay, label, segment, highlight or otherwise modify either an entire image or particular features of an image (e.g., anatomical features or manmade devices within the image). In some instances, the light source 520 may be part of the processing system 170, or vice-versa, or the light source 520 may be controlled (whether directly or indirectly) by the processing system 170.

Any of the CAT scan system 150 or the fluoroscopy system 160, or other related imaging systems capable of imaging anatomy and devices inside the body, may be referred to as an imaging system. Other operations of the CAT scan system 150, the fluoroscopy system 160, the processing system 170, the light source 520, and the intraluminal device 402 will be described below in greater detail.

FIG. 1B is a diagrammatic, schematic representation of at least a portion of an intraluminal stent graft fenestration system 100, in accordance with aspects of the present disclosure. The intraluminal stent graft fenestration system 100 includes a functional catheter 120 (e.g., a laser catheter, balloon catheter, glue injection catheter, intravascular ultrasound (IVUS) catheter, etc.) that can be inserted into, and advanced through, a blood vessel or other body lumen 110. Depending on the implementation, the catheter 120 may be guided by a steerable sheath or guide catheter 130, or by a guidewire 140. The guidewire 140 may for example be a Fiber Optic Real Shape (FORS) guidewire, a sensing guidewire, or other type of guidewire. Any of the catheter 120, guide catheter 130, or guidewire 140 may be radiopaque or may include radiopaque features, such that they can be imaged clearly by a CAT scan system 150 and/or a fluoroscopy x-ray system 160, under the control of one or more processors of the processing system 170. Any of the catheter 120, guide catheter 130, or guidewire 140 may be considered an intravascular or intraluminal device 402 comprising a flexible elongate member 421 (see FIG. 1).

Before continuing, it should be noted that the examples described above are provided for purposes of illustration, and are not intended to be limiting. Other devices and/or device configurations may be utilized to carry out the operations described herein.

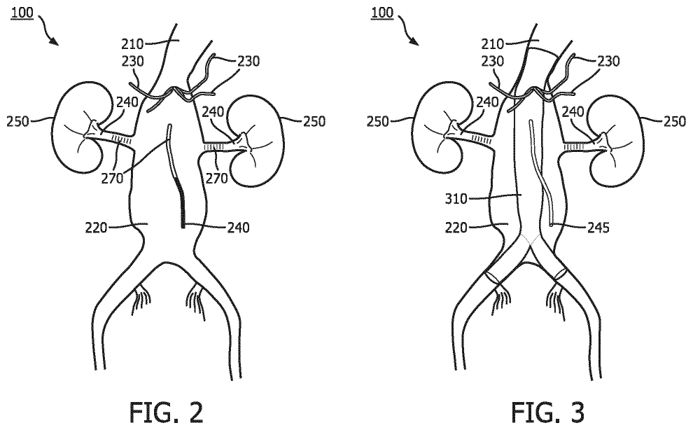

FIG. 2 is a diagrammatic, schematic representation of at least a portion of an intraluminal stent graft fenestration system 100, in accordance with aspects of the present disclosure. Visible is the aorta 210, which (in the example shown in FIG. 2) includes an aneurysm 220. The aneurysm 220 can be a dangerous pathological condition that may for example result from illness or injury. A number of sacrificial branch arteries 230 extend from the aorta 210. The sacrificial branch arteries 230 will be covered by the stent graft and will therefore be blocked from carrying blood to other portions of the body. Also extending from the aorta 210 are a number of critical branch arteries 240, including the renal arteries 240 which supply blood to the kidneys 250. Blood supply through the critical branch arteries 240 may be considered important for the health of the patient, and therefore stent graft branches will be supplied for these critical branch arteries 240.

In some aspects, primary stenting of the critical branch arteries 240 may take place prior to placement of the aortic stent graft. Thus, a stent 270 may be placed at the end of each critical branch artery 240. The stents 270 may for example be radiopaque, such that they can effectively mark both the locations and the diameters of the critical branch arteries 249 within the aortic aneurysm 220 on both planning images (e.g., 3D CAT scan images) and real-time surgical images (e.g., fluoroscopic x-ray images).

FIG. 3 is a diagrammatic, schematic representation of at least a portion of an intraluminal stent graft fenestration system 100, in accordance with aspects of the present disclosure. An endovascular exclusion step occurs when the main stent graft 310 is positioned within the aorta 210 to relieve pressure on the aneurysm 220. At this stage, the main stent graft 310, blocks blood flow to both the sacrificial branch arteries 230 and the critical branch arteries 240. To limit the chance of ischemic injury to body tissues such as the kidneys 250, the surgeon may need to limit the amount of time the critical branch arteries 240 remain blocked.

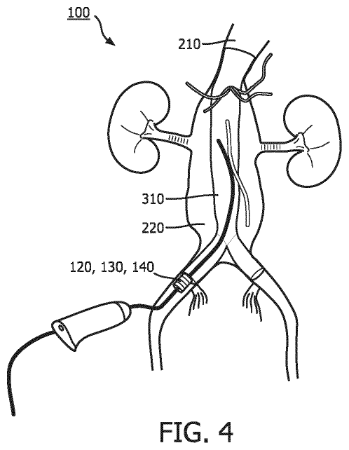

FIG. 4 is a diagrammatic, schematic representation of at least a portion of an intraluminal stent graft fenestration system 100, in accordance with aspects of the present disclosure. To aid in fenestration, a guidewire 140 or a steerable sheath or guide catheter 130 may be advanced into the main stent graft 310 within the aneurysm 220 of the aorta 210, until the distal end of the guidewire 140 or steerable sheath 130 approaches a desired puncture point. A functional catheter 120 can then be advanced over the guidewire 140 or within the guide catheter 130 until it also reaches the desired puncture point. In some aspects (e.g., if the functional catheter 120 is itself steerable), the functional catheter 120 may be advanced into the main stent graft 310 without a guidewire 140 or steerable sheath 130.

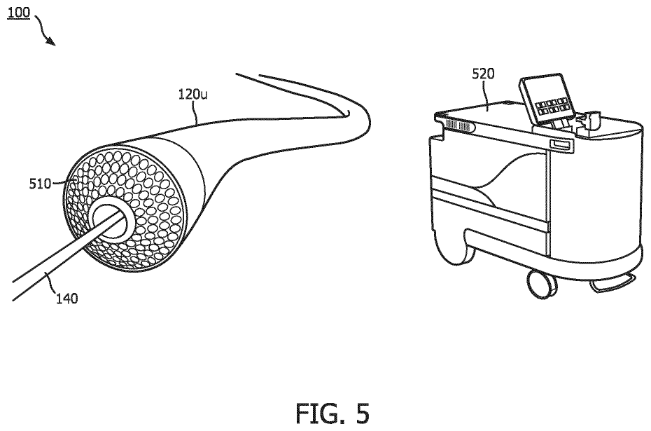

FIG. 5 is a diagrammatic, schematic representation of at least a portion of an intraluminal stent graft fenestration system 100, in accordance with aspects of the present disclosure. The functional catheter 120 may for example be a laser catheter 120u that includes a plurality of optical fibers 510, and that is capable of puncturing the side wall material of the main stent graft. In an example, the light source 520 of the laser catheter 120u is a commercially available medical excimer laser from Philips, and the laser catheter 120u is a commercially available Turbo-Elite laser atherectomy catheter, or other Philips atherectomy laser. Excimer is an abbreviation of “excited dimer” and describes a laser technology that relies on the use of a combination of a rare noble gas (helium or xenon) and a halide (bromine or chlorine). The laser light produced may for example be in the ultraviolet range (about 300 nm), have a high energy, and have a maximum absorption depth of 50 μm. This specificity may allow the targeted material to be vaporized to a limited depth, without risking injury to structures beyond.

Excimer lasers have found wide medical application in fields including ophthalmologic surgery (particularly refractive surgery), interventional cardiology (to facilitate the extraction of pacemaker leads and the recanalization of fibrous and atheromatous occlusive lesions in the coronary arteries), and vascular surgery. Vascular surgeons have used laser catheters 120u at the femoropopliteal level for the treatment of chronic stenoses and occlusions as well as in treating in-stent restenosis. However, laser catheters 120u can also be used to puncture (fenestrate) a primary aortic stent graft, to facilitate placement of branch stent grafts into critical branch arteries.

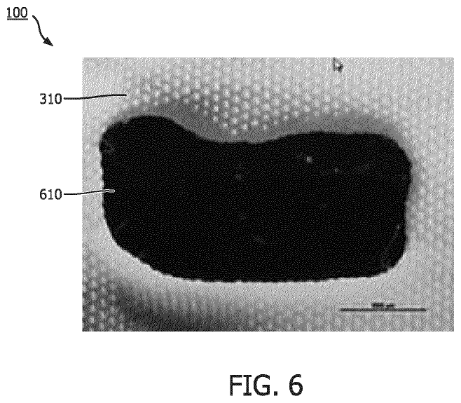

FIG. 6 is a diagrammatic, schematic representation of at least a portion of an intraluminal stent graft fenestration system 100, in accordance with aspects of the present disclosure. After a brief pulse at a present fluence (e.g., 60 fluence, 45 Hz for 3 seconds) from the laser catheter 120u, the material of the stent graft 310 may be fenestrated with a hole 610. Depending on the implementation, the hole 610 may subsequently be circularized and/or enlarged.

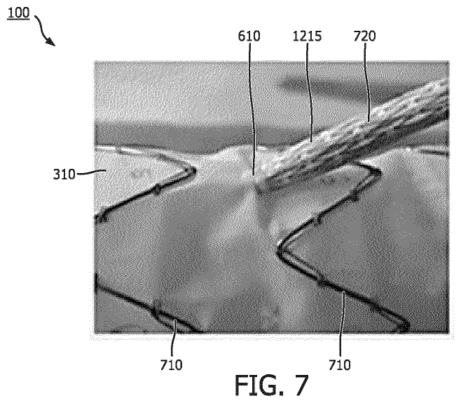

FIG. 7 is a diagrammatic, schematic representation of at least a portion of an intraluminal stent graft fenestration system 100, in accordance with aspects of the present disclosure. FIG. 7 shows a bench test experiment on laser fenestration. Visible are the main stent graft 310 and its support struts 710. The struts 710 may for example be metallic or polymer wires attached to the fabric of the main stent graft 310. In the example shown in FIG. 7, the hole or fenestration 610 is positioned such that it does not overlap with, and is not interfered with by, the stent struts 710. A branch artery stent graft 720 can then be placed through the hole or fenestration 610, to provide a connection between the main stent graft 310 and the critical branch artery, thus ensuring blood flow to tissues such as the kidneys. In an example, the branch artery stent graft 720 is held in place by friction in the hole or fenestration 610. Thus, it may be desirable for the size of the hole or fenestration 610 to closely match the diameter of the branch artery stent graft 720.

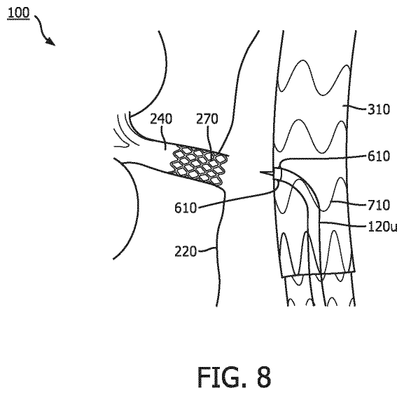

FIG. 8 is a diagrammatic, schematic representation of at least a portion of an intraluminal stent graft fenestration system 100, in accordance with aspects of the present disclosure. The main stent graft 310 is punctured by the laser catheter 120u, in such a way that the hole or fenestration 610 is well spaced from the stent struts 710, and such that the hole or fenestration 610 is aligned with the critical branch artery 240, which may be marked with a radiopaque critical branch artery stent 270. Also visible is the aneurysm 220. In present LfEVAR procedures, placement and alignment of the hole or fenestration 610 with the critical branch artery 240 may rely heavily on the judgment and dexterity of the surgeon, with attendant risks as described above. The present disclosure provides clear guidance to the surgeon for this step, as described below.

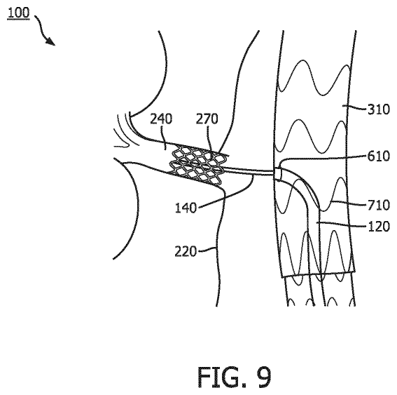

FIG. 9 is a diagrammatic, schematic representation of at least a portion of an intraluminal stent graft fenestration system 100, in accordance with aspects of the present disclosure. In the example shown in FIG. 9, a guidewire (e.g, a 0.014-inch diameter wire) is advanced through the hole or fenestration 610 in the main stent graft 310, such that it enters the critical artery branch 240 at the location of the marking stent 270, and can thus be used to guide additional stents or catheters into the branch artery 240.

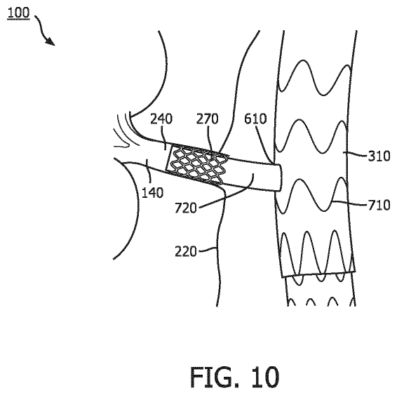

FIG. 10 is a diagrammatic, schematic representation of at least a portion of an intraluminal stent graft fenestration system 100, in accordance with aspects of the present disclosure. In the example shown in FIG. 10, a branch artery stent graft 720 is deployed through the hole or fenestration 610, through the marking stent 270, and into the branch artery 240, thus ensuring blood flow through the branch artery 240 to other tissues in the body. Also visible are the stent struts 710 and aneurysm 220.

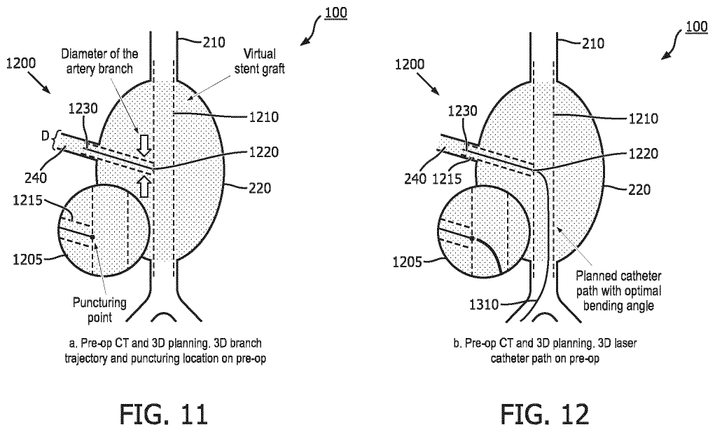

FIGS. 11-15 will now show the steps for improving the planning and laser catheter guidance during laser fEVAR procedures.

FIG. 11 is a diagrammatic, schematic representation of a CT or CAT scan planning image 1200, and magnified portion 1205 of the planning image 1200, showing at least a portion of an intraluminal stent graft fenestration system 100, in accordance with aspects of the present disclosure. Once the CT planning image 1200 has been captured (e.g., by the CAT scan imaging system 150 of FIG. 1), the planning image 1200 may for example show the aorta 210, the aortic aneurysm 220, and at least one critical branch artery 240. However, the present disclosure provides for helpful annotations of the planning image 1200. The annotations may for example include a virtual stent graft 1210, indicating the dimensions and desired positioning of the main stent graft 310 within the aorta 210 to block off the aneurysm 220. In addition, an annotation may be shown that extends virtual sidewalls 1215 of diameter D, and a virtual centerline or branch artery trajectory 1230, of the branch artery 240 from the actual sidewalls and centerline of the branch artery 240 until they intersect with the virtual stent graft 1210. The point where the virtual centerline 1230 intersects the virtual stent graft 1210 then becomes the desired puncture point 1220, where it is most favorable for the laser catheter 120u to puncture the main stent graft 310 (as shown for example in FIG. 8). Such annotations to the planning CT image 1200 may be extremely useful to the surgeon in planning and executing the laser fEVAR procedure, particularly when the branch arteries 240 are not marked with stents.

FIG. 12 is a diagrammatic, schematic representation of a CT planning image 1200 and magnified portion 1205 showing at least a portion of an intraluminal stent graft fenestration system 100, in accordance with aspects of the present disclosure. The optimal 3D path 1310 of the laser catheter may be computed such that the path is contained inside the aorta 210 and the virtual stent graft 1210, and approaches the desired puncture point 1220 as orthogonally as possible, without bending beyond a specified bend radius (dependent on the specific type of catheter being used). Depending on the implementation, the trajectory of the laser catheter may be computed for each artery branch the surgeon intends to fenestrate.

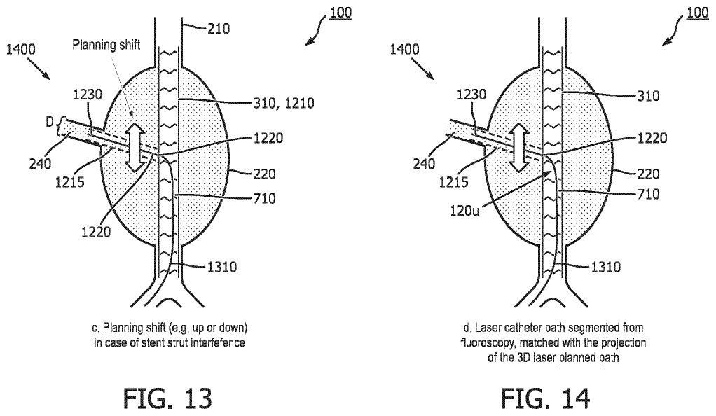

FIG. 13 is a diagrammatic, schematic representation of a fluoroscopy image 1400 showing at least a portion of an intraluminal stent graft fenestration system 100, in accordance with aspects of the present disclosure. A 2D projection of the surgical plan annotations from the planning CT image 1200 may be registered onto the fluoroscopy image 1400 in real time or near-real time, such that, for example, the annotations move along with the associated anatomical features in response to heartbeat, respiration, etc. These annotations may for example include the virtual stent graft 1210, virtual sidewalls 1215, virtual centerline 1230, desired puncture point 1220, and desired catheter path 1310.

During the procedure, the main stent graft 310 is placed in the aorta 210, aligned with the virtual stent graft 1210, to seal off the aneurysm 220. After that the main stent graft 310 is deployed, the C-arm angle for the targeted artery branch is selected and the surgical plan is projected in 2D on the new C-arm angle. On the fluoroscopy, the position of the stent graft 310 and its stent struts 1210 are visible.

At this point, differences between the planned landing site and the actual landing site of the main stent graft 310 stent are visible, along with the final positions of the stent struts, and the planning can be adjusted accordingly. For example, the puncture locations and the laser catheter paths may be adjusted to avoid the stent struts 710. As noted above, the stent struts may interfere with the desired puncturing position of the laser catheter, since a hole cannot be opened in correspondence of the struts. Therefore, the 3D puncturing position 1220 can be shifted, for example above or below the stent struts, in order to reduce interference. Such shifting may for example occur automatically, with an option for the surgeon to override the position and select a different adjustment. Once the puncture point 1220 has been moved, the corresponding artery branch trajectory 1230 and planned laser catheter path 1310 can be shifted or adjusted, and the bend radius of the laser catheter can be recalculated (e.g., in real time or near-real time) based on the updated path, to ensure it remains within its operating specifications, and the resulting catheter path annotations can be updated on the fluoroscopy image 1400.

If the stent strut is not exactly on the planned puncturing location of the laser, but still in the nearby area inside the projected diameter of the targeted vessel to be fenestrated, the stent struts may put some pressure on the branch stent graft once it is deployed and cause its closure (or partial closure). In this case, the planned puncturing position can also be shifted.

FIG. 14 is a diagrammatic, schematic representation of a fluoroscopy image 1400 showing at least a portion of an intraluminal stent graft fenestration system 100, in accordance with aspects of the present disclosure. In an example, the system segments the actual path of the laser catheter 120u on the fluoroscopy image 1400, and compares it against the 2D projection of the 3D laser planned path 1310.

The laser catheter 120u (e.g. used with a steerable sheath) is advanced inside the main stent graft 310 until the planned puncturing point 1220 is reached. The laser catheter 120u is guided by the 3D planned trajectory 1310, which is projected in 2D on the fluoroscopy image. The catheter 120u is segmented from the fluoroscopy image (e.g. by image-based segmentation) and matched with the projected planned path in 2D, such that the catheter position can be adjusted by the surgeon in order to match the projected plan. In some aspects, the average distance between the planned path 1310 and the segmented position of the catheter 120u is computed and displayed, to assess how well the laser catheter 120u overlays the planned 3D path 1310. The surgeon may then be notified by the system (e.g. by a message on the display) in case the catheter 120u needs adjustment, or for example in case the angle of the catheter 120u exceeds the maximum specified bend radius for that catheter type. In other aspects, a range of acceptable paths 1310 can be displayed, and the surgeon may then position the catheter 120u inside that range. After the surgeon places the laser catheter 120u in the correct position, the laser can then be activated for a period of time (e.g., 3 seconds) in order to puncture the main stent graft 310 at the desired puncture point.

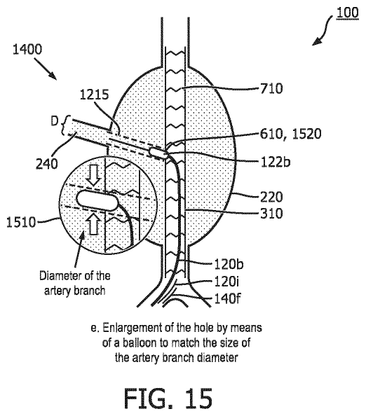

FIG. 15 is a diagrammatic, schematic representation of a fluoroscopy image 1400 and enlarged portion 1510, showing at least a portion of an intraluminal stent graft fenestration system 100, in accordance with aspects of the present disclosure. Enlargement of the hole 610 (e.g., by means of a balloon catheter 120b equipped with a cutting balloon, scoring balloon, or other type of balloon 122b) can match the diameter of the enlarged hole 1520 to the diameter D of the branch artery, as shown on the fluoroscopy image 1400 by the virtual vessel walls 1215 as they intersect with the main stent graft 310. Afterwards, a guidewire can be advanced through the enlarged hole 1520 in the main stent graft 310 to reach the artery branch 240, so that a branch stent graft can be placed in the artery branch 240 as described above.

IVUS for Intravascular guidance

In other aspects, an IVUS catheter 120i (or OCT catheter, or combinations thereof, or other intravascular imaging device) may be advanced together with the laser catheter 120u, and can be used to better locate the stent struts 710 before puncturing the main stent graft 310 with the laser. Also, after graft fenestration with the laser, the IVUS catheter 120i can be advanced inside the hole 610 or 1520 in the main stent graft 310 to visualize the origin of the artery branch 240 and guide the branch stent graft deployment into the artery branch 240.

FORS for improved laser catheter guidance

In still other aspects, the laser catheter 140u may be guided by a FORS guidewire 140f, which can be registered on the fluoroscopy image 1400 and used to track, in 3D and in real time or near-real time, the path of the laser catheter 120u as it moves toward the desired puncture point 1220. In some aspects, the 3D planned path 1310 for the laser catheter 120u is projected in 2D on the fluoroscopy image 1400, and the path of the laser catheter 120u is segmented from the fluoroscopy (meaning, the segmented path is in 2D). By using the FORS guidewire 140f, the path of the laser catheter 120u can be visualized in 3D on the fluoroscopy image 1400, and can be adjusted in order to match the 3D planned path 1310, potentially improving navigation accuracy.

When using the FORS guidewire 140f in combination with the laser catheter, the FORS device can track in 3D the positions at which the laser is pulsed. This can help prevent the laser from damaging tissue near the stent graft, and can be used to create a 3D map of the laser fenestration locations.

Improved visualization of artery branches and main stent graft

In still other aspects, pre-stenting of the artery branches 240 is done, thus to improve visualization of the arteries 240 in the fluoroscopy. The stents 270 of the artery branches 240 (see FIG. 2) can be segmented from the fluoroscopy image 1400 (e.g. via image based segmentation), and used for updating the planned trajectory 1310 of the laser catheter 120u based on the locations of the artery branches 240 on each new fluoroscopy image 1400. Furthermore, once the main stent graft 310 is deployed, the same approach can be used for updating the surgical planning with the correct positioning of the main stent graft 310. This allows for updating the 3D puncturing point 1220, according to the new position of the artery branch 240 and the deployed main stent graft 310. This method may for example be useful in compensating for artery deformation that may occur during the procedure, and misalignment of the surgical plan annotations on top of the fluoroscopy image 1400.

Beside stents, other fiducials (e.g., radiopaque markers) can be placed in the artery branches 240 for improving their localization in the fluoroscopy image 1400. In another case, the artery branches may be cannulated before the main stent graft 310 is deployed.

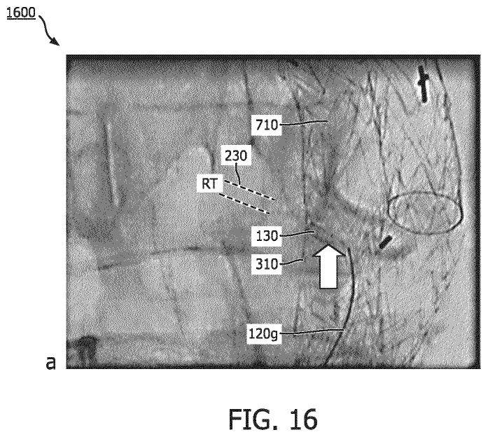

FIG. 16 is a fluoroscopy image 1600 of an aortic stent graft 310, outside of which an endoleak has formed in an aortic side branch artery, in accordance with aspects of the present disclosure. The approximate position of the side branch artery has been marked with dashed lines 230. This is shown here purely for explanatory purposes; in traditional endoleak repair procedures, such annotations to the fluoroscopy image 1600 may not be available. Also visible are a glue catheter, injection catheter, or glue injection catheter 120g, stent struts 710, and mammary catheter, guide catheter, or steerable sheath 130.

Type II endoleaks can form in sacrificial branch arteries (e.g., arteries blocked by the main stent graft 310) after endovascular repair of abdominal aortic aneurysms. The endoleak may for example result from retrograde flow through the sacrificial branch arteries, refilling the aneurysm sac. Such endoleaks can be complex vascular structures that contain an endoleak cavity, or nidus, with several feeding and draining vessels, similar to an arteriovenous malformation. Most such endoleaks are transient and either resolve spontaneously within a few months or remain benign. However, persistent type II endoleaks can be associated with expansion of the aortic aneurysm sac, and therefore may require secondary interventions to avoid rupture. Although open and laparoscopic techniques have been described to eliminate side branch perfusion, endovascular methods may be preferred, given their minimally invasive nature.

Endovascular methods include transarterial embolization (TAE), translumbar embolization (TLE), and, more recently, transcaval embolization (TCE). TAE may be the most commonly used method, and its technical success may require catheter and guidewire manipulations through small and tortuous arteries, which can be technically challenging. TLE may not always be feasible, owing to the location of the endoleak relative to the inferior vena cava, bowel loops, or kidney, or its location in the pelvis, where safe needle access may be blocked by surrounding bony structures. TCE has been reported to be effective, particularly in patients who have no transarterial access and are not candidates for TLE. As an alternative to transarterial, translumbar, and transcaval approaches, transgraft embolization (TGE) technique can be used.

Transgraft embolization (TGE) uses laser energy to micropuncture the endograft via a transfemoral arterial approach to access the aneurysm sac at the precise site of the type II endoleak nidus, regardless of its anatomic location.

Technique for Laser trans-graft embolization

The main goal may be to open a small hole in the fabric of the already deployed stent graft 310 using a laser device in order to advance a catheter into the aneurism sac to obliterate the type II endoleak at the level of the nidus.

A planning computed tomography (CT) angiography may be performed in order to assess the diameter of the aneurysm sac and the presence of the type II endoleak, e.g., the retrograde flow from one or more artery branches. The CT angiography may be analyzed to determine from which limb (e.g., which leg) the endoprosthesis can be accessed. Following percutaneous catheterization of the femoral artery, a sheath may be advanced over a standard guidewire of choice. Through the sheath, a guide catheter can be advanced to the level of the endoleak.

In the example shown in FIG. 16, a 6-F internal mammary catheter 130 is positioned so that it is pointed toward the known type II endoleak.

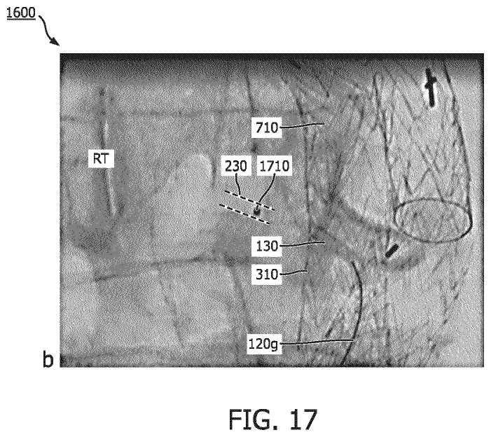

FIG. 17 is a fluoroscopy image 1600 of an aortic stent graft 310, outside of which an endoleak has formed in an aortic side branch artery (marked with dotted lines 230), in accordance with aspects of the present disclosure. In the example shown in FIG. 17, the stent graft 310 is punctured using a 0.9-mm coronary laser probe (e.g. Turbo-Elite laser atherectomy catheter, Philips), pointed toward the site of the endoleak. The laser may for example be activated at a frequency of 60 pulses/s and fluency of 60 mJ/mm². A Turbo-Elite catheter may be used in some cases. A radiopaque marker 1710 is located on the distal end of the laser catheter, now seen through the endograft, inside the abdominal aortic aneurysm sac (arrow). The guide catheter 130 or a guidewire 140 can then be advanced into the side branch artery 230.

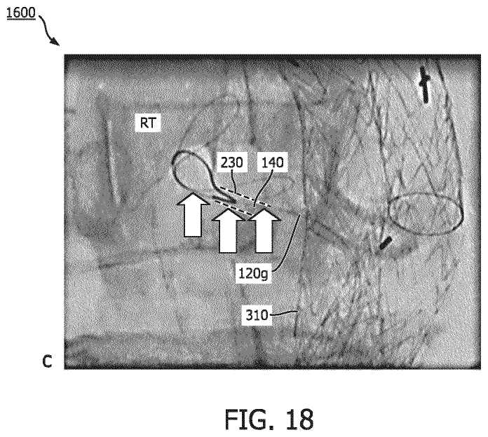

FIG. 18 is a fluoroscopy image 1600 of an aortic stent graft 310, outside of which an endoleak has formed in an aortic side branch artery (marked with dotted lines 230), in accordance with aspects of the present disclosure. A guidewire (e.g., a 0.014-inch guidewire) 140 has been advanced into the aneurysm sac and then exchanged for the glue injection microcatheter 120g.

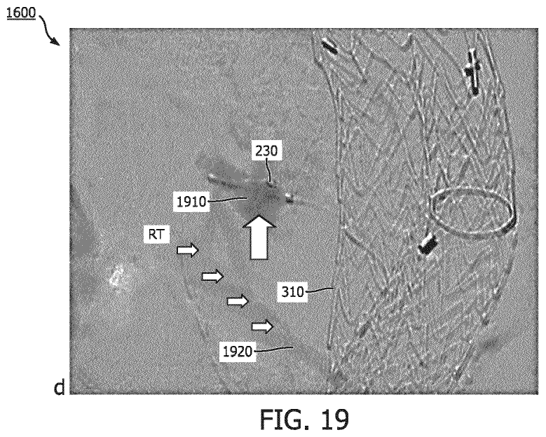

FIG. 19 is a fluoroscopy image 1600 of an aortic stent graft 310, outside of which an endoleak has formed in an aortic side branch artery (marked with dotted lines 230), in accordance with aspects of the present disclosure. In the example shown in FIG. 19, selective digital subtraction angiography has been performed to show the endoleak. Digital subtraction angiography may for example be obtained via exchanged 2.4-F microcatheter (e.g., Philips Echelon) over the guide wire into the aneurysm sac. Note the nidus of the type II endoleak 1910 and vascular structure 1920, most likely representing lumbar arteries.

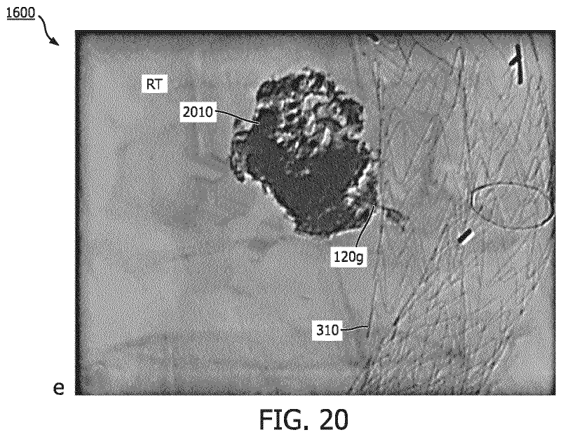

FIG. 20 is a fluoroscopy image 1600 of an aortic stent graft 310, outside of which an endoleak has formed in an aortic side branch artery, in accordance with aspects of the present disclosure. Through the glue injection microcatheter 120g, a therapeutic material 2010 (e.g., Onyx glue from Medtronic, or a similar medical adhesive compound) can be administered to obstruct the type II endoleak at the level of the nidus. This x-ray fluoroscopy image was acquired after administration of the therapeutic material 2010 through the glue injection microcatheter 120g. Exact positioning of the glue catheter 120g, and the exact amount of therapeutic material 2010 to inject, are typically left to the subjective judgment of the surgeon. Thus, present procedures and systems may include few safeguards against injecting too much or too little adhesive.

In some embodiments, the therapeutic material 2010 may be or include a medical adhesive as described above, but may also be or include a solvent, a drug, a contrast material, a solution of cells (e.g., platelets), or other material deemed important or desirable by the surgeon.

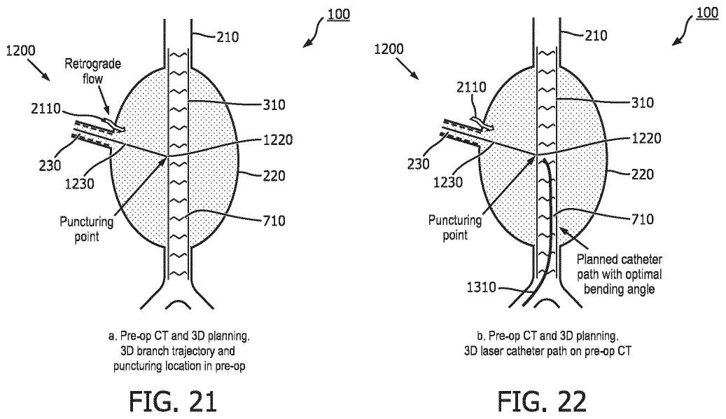

FIGS. 21-27 will now show the steps for improving the planning, laser catheter guidance, glue catheter guidance, and glue injection during laser endoleak repair procedures. The present disclosure describes a method for improving the navigation during the Laser transgraft embolization technique (endoleak type II repair). In particular, the improved navigation is provided by:

Pre-planning the puncturing point on the stent graft in 3D;

Planning the minimum length of the vessel with retrograde flow that needs to be filled, and the volume of glue that needs to be injected;

Defining of the catheter laser path in 3D during the surgical planning;

Adjusting the pre-plan before puncturing, in order to avoid the stent struts during laser perforation;

On the fluoroscopy image, segmenting the laser catheter to assess whether the catheter follows the 3D planned trajectory;

Advancing the glue catheter in the vessel until it passes the minimum vessel portion to be filled; and

Injecting glue until the entire portion of the vessel is filled, while the glue catheter is retracted.

FIG. 21 is a diagrammatic, schematic representation of a CT planning image 1200 showing at least a portion of an intraluminal stent graft fenestration system 100, in accordance with aspects of the present disclosure. Visible are the aorta 210, aneurysm 220, stent graft 310, stent struts 710, and blocked (sacrificial) branch artery 230. Retrograde blood flow 2110 through the branch artery 230 creates the endoleak condition. However, the present disclosure provides annotations to the CT panning image 310, including a virtual centerline 1230 that extends from the centerline of the branch artery 230 until it intersects with the stent graft 310, where it designates the desired puncture point 1220.

During the pre-planning, a pre-operative CT angiography 1200 (or similar 3D image of the patient) may be used for planning the procedure. The vessels 210, 230 and the stent graft 310 in the pre-op CT image may be segmented and used as a roadmap for the planning. Optimal C-arm angles may be defined on the pre-op CT for each artery with retrograde flow. These angles can be acquired pre-operatively by examining the anatomical configuration or simulating fluoroscopic views through the CT volume (for instance, using digitally reconstructed radiographs).

The artery branch 230 with retrograde flow 2110 may be segmented and the trajectory 1230 of the central axis of the branch artery 230 defined. The targeted vessel trajectory 1230 can then then be prolonged to intersect with the main stent graft 310, and their intersection defines the 3D position of the desired puncture point 1220 of the main stent graft. As an alternative, instead of prolonging the artery trajectory, intersection with the main stent graft can be achieved with other related methods, such as projecting the origin of the branch artery 230 orthogonally to the axis of the main stent graft 310.

In another case, where multiple arteries with retrograde flow need embolization, a puncturing point can be used for more than one artery to minimize the number of holes in the fabric of the stent graft. As an example, a puncturing point for multiple arteries can be selected as: One of the points among the already defined points, that minimizes the distance between the stent graft 310 and the origins of the selected arteries 230 that need embolization, or: A new point that minimizes distance between the stent graft and the origins of the selected arteries 230 that need embolization. Besides the methods described just before, still other other metrics can be used for choosing the desired puncture point 1220 on the stent graft 310.

FIG. 22 is a diagrammatic, schematic representation of a CT planning image 1200 showing at least a portion of an intraluminal stent graft fenestration system 100, in accordance with aspects of the present disclosure. Visible are the aorta 210, aneurysm 220, stent graft 310, stent struts 710, blocked (sacrificial) branch artery 230, retrograde blood flow 2110, and desired puncture point 1220. In this step, as described above, the optimal 3D path 1310 of the laser catheter can be computed, such that the path is contained inside the 3D vessel roadmap segmented from the pre-op CT 1200, such that the laser burns a fairly circular (as opposed to elliptical) hole, and such that the path prevents the laser catheter from bending more than its specified minimum bend radius. This specified minimum depends on the type of catheter being used. Depending on the implementation, the trajectory of the laser catheter is computed for each desired puncture point 1220 previously identified.

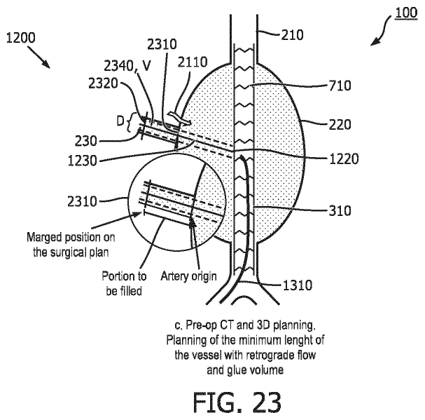

FIG. 23 is a diagrammatic, schematic representation of a CT planning image 1200 and magnified region 2310, showing at least a portion of an intraluminal stent graft fenestration system 100, in accordance with aspects of the present disclosure. Visible are the aorta 210, aneurysm 220, stent graft 310, stent struts 710, blocked (sacrificial) branch artery 230, retrograde blood flow 2110, and desired puncture point 1220. In this step, the portion 2340 of the artery branch 230 that will be filled with glue can be planned on the pre-op CT image 1200. The portion 2340 to be filled may for example be defined by the surgeon (for example, a 5 cm length of the artery branch 230) and goes from the origin of the artery branch 230 (located at the aneurysm sac) and moves distally from there. The distal position 2320 on the artery branch 230 that represents the minimum portion to be filled can then be marked on the surgical planning image 1200, along with the proximal position 2310 at the origin of the artery branch 230. The volume V of this portion 2340 of the artery branch 230 represents the required volume of glue that is going to be used. Several methods can be used for computing the volume of glue. The volume V can be easily calculated by looking at the length of the portion to be filled and at its diameter D or its known cross-sectional area. In another case, the volume V may be computed by counting the number of voxels in the pre-op CT that represent the artery portion 2340 that needs to be filled.

In other aspects, the volume of glue to be injected in the artery may be predefined, and given a pre-op CT image or anatomical model, the spatial portion of the artery to be filled can be computed. For example, the length of the portion 2340 of artery to be filled may be computed by dividing the volume of glue by the cross-sectional area of the artery. In another case, the portion of artery 2340 may be computed by counting the number of voxels of the segmented artery branch, starting from the origin of the artery branch 230 at the aneurysm sac, until the defined volume of glue is reached.

The portion 2340 of the artery branch represents only the minimum length of the artery to be filled. The surgeon can fill a much larger portion, if needed.

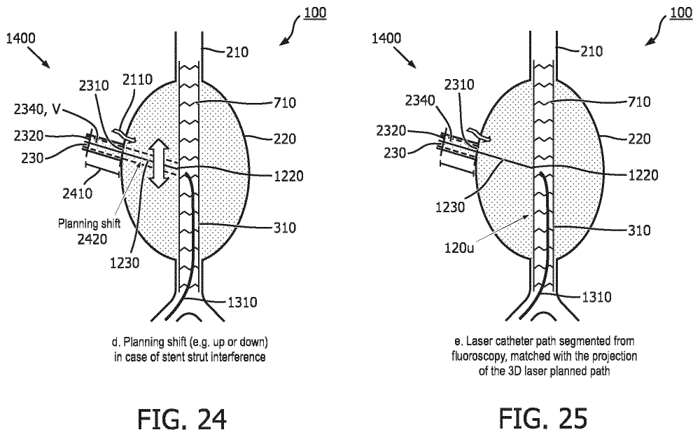

FIG. 24 is a diagrammatic, schematic representation of a fluoroscopy image 1400 showing at least a portion of an intraluminal stent graft fenestration system 100, in accordance with aspects of the present disclosure. Visible are the aorta 210, aneurysm 220, stent graft 310, stent struts 710, blocked (sacrificial) branch artery 230, retrograde blood flow 2110, desired puncture point 1220, and the distal volume marker 2320 and proximal volume marker 2340, which define the portion 2340 or volume V of the branch artery 230 that is to be filled with adhesive.

As the surgical procedure starts, the 3D surgical planning annotations made on the pre-op CT 1200 are registered on the patient. A 2D projection of the surgical planning annotations can be overlaid on the 2D fluoroscopy image, such that the surgeon can see the branch artery centerline 1230, desired puncture point 1220, desired path 1310 for the laser catheter, and the distal volume marker 2320 and proximal volume marker 2340, which define the portion 2340 or volume V. On the fluoroscopy image 1400, the position of the stent graft 310 and its stent struts 710 will also generally be visible to some degree.

As described above, the stent struts may interfere with the desired puncturing position 1220 of the laser catheter, since it may not be feasible or desirable to open a hole through the location of a strut 710. Therefore, the 3D puncturing position 1220 can be shifted, for example above or below the stent struts, in order to reduce interference. Such shifting may for example occur automatically, with an option for the surgeon to override the position and select a different adjustment. A change in location for the desired puncture point 1220 may result in automatic recalculation of the desired catheter path 1310, ad described above.

FIG. 25 is a diagrammatic, schematic representation of a fluoroscopy image 1400 showing at least a portion of an intraluminal stent graft fenestration system 100, in accordance with aspects of the present disclosure. Visible are the aorta 210, aneurysm 220, stent graft 310, stent struts 710, blocked (sacrificial) branch artery 230, retrograde blood flow 2110, desired puncture point 1220, and the distal volume marker 2320 and proximal volume marker 2340, which define the portion 2340 or volume V of the branch artery 230 that is to be filled with adhesive.

The laser catheter 120u (e.g. used with a steerable sheath or guidewire) may be advanced inside the main stent graft 310 until the planned puncturing point 1220 is reached. The laser catheter 120u can be guided by the 3D planned trajectory 1310, projected in 2D on the fluoroscopy image 1400. The catheter 120u may be segmented from the fluoroscopy image 1400 (e.g. by image-based segmentation) and matched with the projected planned path in 2D, such that the catheter position can be adjusted (e.g., by the surgeon) in order to match the planned path 1310. In some aspects, the average distance between planned path 1310 and segmented position of the catheter 120u is computed, to assess how well the laser catheter 120u overlays the planned or optimized 3D path 1310. The surgeon may be notified by the system (e.g. by a message on the display) in case the catheter needs adjustment, or for example in case the angle of the catheter is tighter than the minimum bending radius. In other aspects, a range of acceptable paths can be displayed, and the surgeon can position the catheter 120u inside that range. After placing the laser catheter 120u in the correct position, the main stent graft 310 can be punctured by the laser, forming a hole or fenestration.

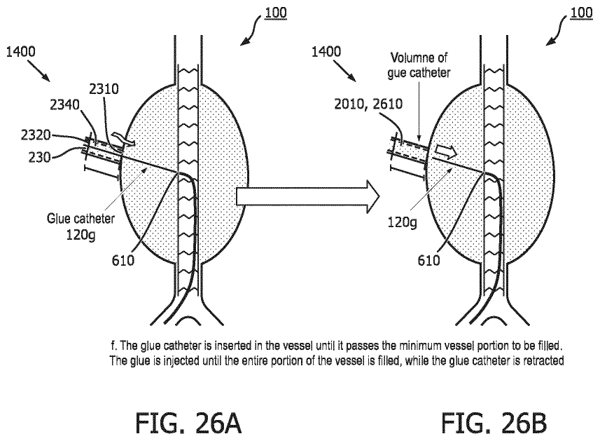

FIG. 26A is a diagrammatic, schematic representation of a fluoroscopy image 1400 showing at least a portion of an intraluminal stent graft fenestration system 100, in accordance with aspects of the present disclosure. Passing through the hole or fenestration 610 in the sent graft 310, the glue catheter 120g may be inserted into the branch artery 230, until the distal end of the glue catheter 120g passes the distal marker 2320, which marks the minimum portion of the vessel to be filled. Glue can then be injected, while the glue catheter 120g is withdrawn.

FIG. 26B is a diagrammatic, schematic representation of a fluoroscopy image 1400 showing at least a portion of an intraluminal stent graft fenestration system 100, in accordance with aspects of the present disclosure. The glue injection catheter 120g may be inserted into the branch artery 230, until it passes the minimum portion of the vessel to be filled, marked on the surgical plan. Once there, the therapeutic material 2010 (e.g., glue) can be injected until the entire fill portion 2340 of the branch artery 230 is filled, while the glue catheter is retracted. Where the therapeutic material 2010 is a glue, it may for example be selected such that it remains liquid within the glue injection catheter 210g, but rapidly cured, hardens, gellates, or increases in viscosity upon contact with blood, such that the glue 2010 forms a plug 2610, largely or completely blocking the branch artery 230, such that retrograde flow through the branch artery cannot create an endoleak.

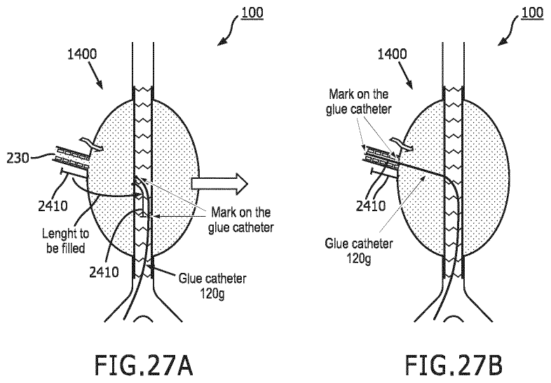

FIG. 27A is a diagrammatic, schematic representation of a fluoroscopy image 1400 showing at least a portion of an intraluminal stent graft fenestration system 100, in accordance with aspects of the present disclosure. FIGS. 27A and 27B demonstrate another method for planning the minimum length of the artery to be filled. In such aspects, the minimum fill length 2410 of the branch artery 230 may be marked on the surgical planning CT image 1200. Subsequently, when the glue catheter 120g is segmented from the fluoroscopy image 1400 (e.g. via image-base segmentation), the minimum fill length 2410 can then be marked or annotated on the image of the glue catheter 120g itself, starting from the distal tip of the glue catheter 120g and moving distally until the minimum fill length 2410 is traversed. Such marking or annotation may take the form of a colored line following the contour of the glue catheter 120g, or a distal marker at the distal end of the glue catheter 120g, plus a proximal marker positioned along the glue catheter at the minimum fill length 2410. Other markings or annotations may be used instead or in addition. In this way, the marked or annotated image of the glue catheter provides direct cues as to how far back in the branch artery 230 the glue vessel needs to be inserted in order to inject the desired volume of glue.

FIG. 27B is a diagrammatic, schematic representation of a fluoroscopy image 1400 showing at least a portion of an intraluminal stent graft fenestration system 100, in accordance with aspects of the present disclosure. In the example shown in FIG. 27B, the glue catheter 120g is advanced inside the artery branch 230 until the marked length 2410 on the glue catheter matches the origin of the artery branch 230. Since the fluoroscopy shows a 2D projection of the glue catheter 120g, the length segmented from the fluoroscopy image 1400 may not represent the real 3D length of the catheter. In some aspects, a FORS guidewire can be used for visualizing the glue catheter 120g in 3D in the fluoroscopy, as described above for the laser catheter 120u.

In other aspects, an IVUS catheter may be advanced together with the laser catheter and is used to better locate the stent struts before puncturing with the laser. Also, after puncturing the graft with the laser, the IVUS catheter can be advanced inside the hole in the main graft to visualize the origin of the artery branch 230 and guide the insertion of the glue catheter 120g into the artery branch.

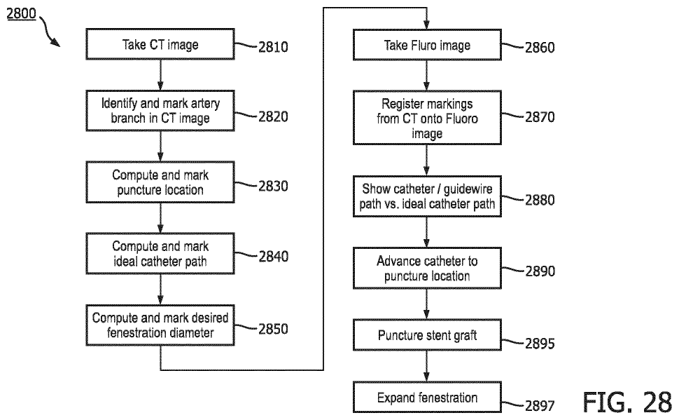

FIG. 28 shows a flow diagram of an example stent graft fenestration method 2800, according to aspects of the present disclosure. It is understood that the steps of method 2800 may be performed in a different order than shown in FIG. 28, additional steps can be provided before, during, and after the steps, and/or some of the steps described can be replaced or eliminated in other aspects. One or more of steps of the method 2800 can be carried by one or more devices and/or systems described herein, such as components of the system 100, processing system 170, and/or processor circuit 3050.

In step 2810, the method 2800 includes capturing a 2D or 3D planning image using a CAT scanner or other imaging technology. Execution the proceeds to step 2820.

In step 2820, the method 2800 includes identifying and marking an artery branch in the planning image. Such identification may for example be done by segmentation algorithms as described in U.S. Pat. No. 9,842,401 to Perval et al., with a priority date of Aug. 8, 2013, and/or U.S. Pat. No. 10,417,765 to Saalbach et al., with a priority date of Jan. 14, 2015, both hereby incorporated by reference as though fully set forth herein. Marking may for example include extending virtual vessel walls and a virtual vessel centerline from the artery branch until they intersect with a real or virtual stent graft in the planning image. Execution then proceeds to step 2830.

In step 2830, the method 2800 includes computing the desired location to puncture the stent graft, and marking this location on the planning image. In some aspects, the desired puncture location is the intersection of the branch artery centerline with the stent graft. Execution then proceeds to step 2840.

In step 2840, the method 2800 includes computing and marking the desired path for the laser catheter. The desired path may, for example, traverse the stent graft such that it meets the desired puncture point at as perpendicular an angle as possible, without exceeding a minimum bend radius. Execution then proceeds to step 2850.

In step 2850, the method 2800 includes computing and marking the desired diameter of the fenestration. In some aspects, this diameter is roughly equal to the diameter of the artery branch, and to the virtual sidewalls of the artery branch extended to meet the stent graft. Execution then proceeds to step 2860.

In step 2860, the method 2800 includes capturing one or more fluoroscopy images of the patient. Execution then proceeds to step 2870.

In step 2870, the method 2800 includes registering the markings from the planning image onto the fluoroscopy images. Such registration may use anatomical landmarks and other information, as described for example in U.S. Pat. No. 8,620,050 to Liao et al., with a priority date of Sep. 23, 2010, and/or U.S. Pat. No. 9,295,435 to Florent et al. with a priority date of Feb. 7, 2011, both hereby incorporated by reference as though fully set forth herein. Execution then proceeds to step 2880.

In step 2880, the method 2800 includes segmenting the laser catheter on the fluoroscopy image, and marking it along with the desired catheter path. In some cases, differences between the actual and desired catheter paths may be numerically computed and reported to the surgeon. Execution then proceeds to step 2890.

In step 2890, the method 2800 includes advancing the laser catheter into the stent graft until it reaches the desired puncture location. In some aspects, such advancement is performed by the surgeon, with guidance from the actual and desired catheter paths shown on the fluoroscopy image. Execution the proceeds to step 2895.

In step 2895, the method 2800 includes activating the laser to puncture or fenestrate the stent graft. In some aspects, the method is now complete. In other aspects, execution then proceeds to step 2897.

In step 2897, the laser catheter is exchanged for a cutting instrument such as a balloon catheter equipped with a cutting balloon, which can enlarge the hole until it meets the desired fenestration diameter. The method is now complete.

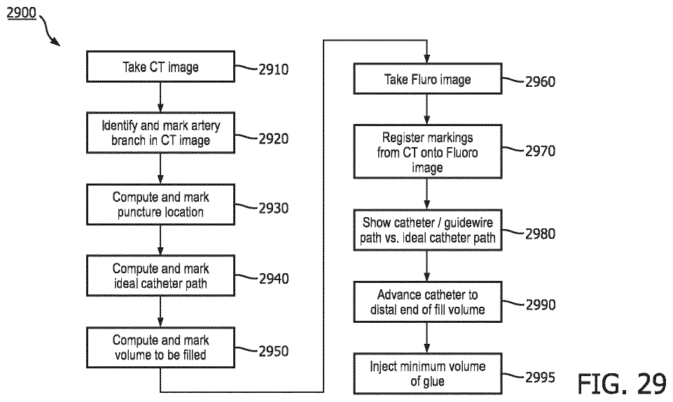

FIG. 29 shows a flow diagram of an example stent graft fenestration method 2900, according to aspects of the present disclosure. It is understood that the steps of method 2900 may be performed in a different order than shown in FIG. 29, additional steps can be provided before, during, and after the steps, and/or some of the steps described can be replaced or eliminated in other aspects. One or more of steps of the method 2900 can be carried by one or more devices and/or systems described herein, such as components of the system 100, processing system 170, and/or processor circuit 3050. In some aspects, the method 2900 may follow or be a continuation of the method 2800 described above.

In step 2910, the method 2900 includes capturing a 2D or 3D planning image using a CAT scanner or other imaging technology. Execution the proceeds to step 2920.

In step 2920, the method 2900 includes identifying and marking an artery branch in the planning image. Such identification may for example be done by segmentation algorithms as described above. Marking may for example include extending virtual vessel walls and a virtual vessel centerline from the artery branch until they intersect with a real or virtual stent graft in the planning image. Execution then proceeds to step 2930.

In step 2930, the method 2900 includes computing the desired location to puncture the stent graft, and marking this location on the planning image. In some aspects, the desired puncture location is the intersection of the branch artery centerline with the stent graft. Execution then proceeds to step 2940.

In step 2940, the method 2900 includes computing and marking the desired path for a glue catheter. The desired path may, for example, traverse the stent graft and stent graft fenestration such that it enters the branch artery. Execution then proceeds to step 2950.

In step 2950, the method 2900 includes computing and marking the volume of the artery branch that will be filled with glue. Such a volume may be computed automatically and marked on the planning image as described above, or may be computed from positions marked by the surgeon on the planning image. Execution then proceeds to step 2960.

In step 2960, the method 2900 includes capturing one or more fluoroscopy images of the patient. Execution then proceeds to step 2970.

In step 2970, the method 2900 includes registering the markings from the planning image onto the fluoroscopy images. Such registration may use anatomical landmarks and other information, as described above. Execution then proceeds to step 2980.

In step 2980, the method 2900 includes segmenting the glue catheter on the fluoroscopy image, and marking it along with the desired catheter path. In some cases, differences between the actual and desired catheter paths may be numerically computed and reported to the surgeon. Execution then proceeds to step 2990.

In step 2990, the method 2900 includes advancing the glue catheter into the artery branch to the distal end of the fill volume. In some aspects, such advancement is performed by the surgeon, with guidance from the actual and desired catheter paths shown on the fluoroscopy image. Execution the proceeds to step 2995.

In step 2995, the method 2900 includes injecting the minimum volume of glue into the artery branch, while withdrawing the glue catheter. In some aspects, such injection is performed by the surgeon, with guidance from the glue volume markings on the fluoroscopy image. The method is now complete.

Flow diagrams and block diagrams are provided herein for exemplary purposes; a person of ordinary skill in the art will recognize myriad variations that nonetheless fall within the scope of the present disclosure. For example, block diagrams may show a particular arrangement of components, modules, services, steps, processes, or layers, resulting in a particular data flow. It is understood that some aspects of the systems disclosed herein may include additional components, that some components shown may be absent from some aspects, and that the arrangement of components may be different than shown, resulting in different data flows while still performing the methods described herein.

Similarly, the logic of flow diagrams may be shown as sequential. However, similar logic could be parallel, massively parallel, object oriented, real-time, event-driven, cellular automaton, or otherwise, while accomplishing the same or similar functions. In order to perform the methods described herein, a processor may divide each of the steps described herein into a plurality of machine instructions, and may execute these instructions at the rate of several hundred, several thousand, several million, or several billion per second, in a single processor or across a plurality of processors. Such rapid execution may be utilized in order to execute the method in real time or near-real time as described herein. For example, when the ideal fenestration point is relocated to avoid a stent strut, recalculation of the ideal catheter path may take place in real time in order to prevent delays that may increase the risk of ischemic injury to the patient.

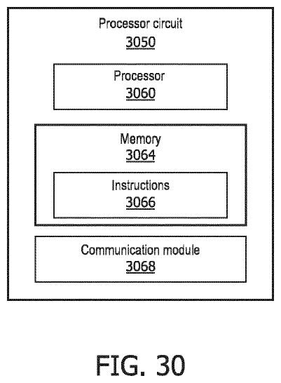

FIG. 30 is a schematic diagram of a processor circuit 3050, according to aspects of the present disclosure. The processor circuit 3050 may be implemented in the stent graft fenestration system 100, the processing system 170, other devices or workstations (e.g., third-party workstations, network routers, etc.), and/or on a cloud processor or other remote processing unit, to implement the method. As shown, the processor circuit 3050 may include a processor 3060, a memory 3064, and a communication module 3068. These elements may be in direct or indirect communication with each other, for example via one or more buses.

The processor 3060 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 3060 may also comprise another hardware device, a firmware device, or any combination thereof configured to perform the operations described herein. The processor 3060 may also be implemented as a combination of computing devices, e.g., a combination of a DSP and a microprocessor, a plurality of microprocessors, one or more microprocessors in conjunction with a DSP core, or any other such configuration.

The memory 3064 may include a cache memory (e.g., a cache memory of the processor 3060), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an aspect, the memory 3064 includes a non-transitory computer-readable medium. The memory 3064 may store instructions 3066. The instructions 3066 may include instructions that, when executed by the processor 3060, cause the processor 3060 to perform the operations described herein. Instructions 3066 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 3068 can include any electronic circuitry and/or logic circuitry to facilitate direct or indirect communication of data between the processor circuit 3050, and other processors or devices. In that regard, the communication module 3068 can be an input/output (I/O) device. In some instances, the communication module 3068 facilitates direct or indirect communication between various elements of the processor circuit 3050 and/or the stent graft fenestration system 100. The communication module 3068 may communicate within the processor circuit 3050 through numerous methods or protocols. Serial communication protocols may include but are not limited to United States Serial Protocol Interface (US SPI), Inter-Integrated Circuit (I²C), Recommended Standard 232 (RS-232), RS-485, Controller Area Network (CAN), Ethernet, Aeronautical Radio, Incorporated 429 (ARINC 429), MODBUS, Military Standard 1553 (MIL-STD-1553), or any other suitable method or protocol. Parallel protocols include but are not limited to Industry Standard Architecture (ISA), Advanced Technology Attachment (ATA), Small Computer System Interface (SCSI), Peripheral Component Interconnect (PCI), Institute of Electrical and Electronics Engineers 488 (IEEE-488), IEEE-1284, and other suitable protocols. Where appropriate, serial and parallel communications may be bridged by a Universal Asynchronous Receiver Transmitter (UART), Universal Synchronous Receiver Transmitter (USART), or other appropriate subsystem.

External communication (including but not limited to software updates, firmware updates, preset sharing between the processor and central server, or readings from the CAT scan device or fluoroscopy imaging device may be accomplished using any suitable wireless or wired communication technology, such as a cable interface such as a universal serial bus (USB), micro USB, Lightning, or FireWire interface, Bluetooth, Wi-Fi, ZigBee, Li-Fi, or cellular data connections such as 2G/GSM (global system for mobiles), 3G/UMTS (universal mobile telecommunications system), 4G, long term evolution (LTE), WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information.

Accordingly, it can be seen that the systems, methods, and devices disclosed herein advantageously improve the ability of a surgeon to plan and execute stent graft fenestrations, while risks and difficulties associated with such procedures. A number of variations are possible on the examples and aspects described above. For example, Fenestration could be achieved with a puncturing instrument other than a laser catheter, including for example a cutting catheter, deployable needle, etc. The glue catheter may be replaced with any sort of injection catheter configured to inject a therapeutic material. The technology described herein may be applied to blood vessels or body lumens other than the aorta, may be applied to fenestration of intraluminal objects other than stent grafts, may be applied to treatment of conditions other than aneurysms, may be applied for fenestrating stent grafts that are not contained inside blood vessels (e.g. bypass grafts), and may be used for the placement of implantable sensors within a blood vessel or other organ.

The logical operations making up the aspects of the technology described herein are referred to variously as operations, steps, objects, elements, components, or modules. Furthermore, it should be understood that these may be performed in any order, unless explicitly claimed otherwise or a specific order is inherently necessitated by the claim language.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the laser fenestration system. Connection references, e.g., attached, coupled, connected, joined, or “in communication with” are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary aspects of the laser fenestration system as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter.

Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. A system for intraluminal fenestration within a body lumen, the system comprising:
a first intraluminal device comprising a flexible elongate member; and
a processor in communication with the first intraluminal device, wherein the processor is configured to:
obtain a planning image from a first imaging system, the planning image comprising the body lumen and a branch lumen extending from the body lumen;
in the planning image, identify a profile of a treatment device;
in the planning image, identify a centerline of the branch lumen, the centerline of the branch lumen extending from the branch lumen to a desired puncture point at a boundary of the profile of the treatment device;
in the planning image, identify a desired trajectory of the first intraluminal device relative to the desired puncture point;
obtain a live procedural image from a second imaging system, the live procedure image comprising the body lumen and the branch lumen extending from the body lumen; and
in the live procedural image, identify the profile of the treatment device, the centerline of the branch lumen, the desired puncture point, the desired trajectory of the first intraluminal device, and an actual trajectory of the first intraluminal device.

2. The system of claim 1, wherein the processor is further configured to:
when the first intraluminal device is within a desired proximity and angle of the desired puncture point, activate a functional portion of the first intraluminal device to puncture the treatment device.

3. The system of claim 1, wherein the processor is further configured to:
in the planning image, identify lumen walls of the branch lumen, extending from the branch lumen to the profile of the treatment device; and
in the live procedural image, identify the lumen walls of the branch lumen, extending from the branch lumen to the profile of the treatment device.

4. The system of claim 1, wherein the processor is further configured to:
after the treatment device is placed within the body lumen, based on a position of the treatment device within the live procedural image, adjust the identified desired puncture point and the identified desired trajectory of the first intraluminal device.

5. The system of claim 1, wherein the processor is further configured to:
display, on the live procedural image, an indication of a difference between the desired trajectory of the first intraluminal device and the actual trajectory of the first intraluminal device.

6. The system of claim 1, further comprising at least one of a guide catheter or a guidewire, wherein the guide catheter or guidewire is configured to affect the actual trajectory of the first intraluminal device.

7. The system of claim 6, wherein the guidewire is a Fiber Optic Real Shape (FORS) guidewire, and wherein the processor is further configured to, as the FORS guidewire moves through the body lumen, identify, on the live procedural image, a 3D trajectory of the FORS guidewire.

8. The system of claim 1, wherein the processor is further configured to:
as a second intraluminal device moves through the body lumen, identify, on the live procedural image, an actual trajectory of the second intraluminal device.

9. The system of claim 8, further comprising at least one of a guide catheter or a guidewire, wherein the guide catheter or guidewire is configured to affect the actual trajectory of the second intraluminal device.

10. The system of claim 9, where the guidewire is a Fiber Optic Real Shape (FORS) guidewire, and wherein the processor is further configured to, as the FORS guidewire moves through the body lumen, display, on the live procedural image stream, a 3D trajectory of the FORS guidewire.

11. The system of claim 8, wherein the second intraluminal device is a cutting device configured to increase a diameter of a puncture in the treatment device.

12. The system of claim 8, wherein the second intraluminal device is an intravascular imaging device.

13. The system of claim 8, wherein the second intraluminal device is an injection catheter configured to inject a therapeutic material.

14. The system of claim 13, wherein the processor is further configured to:
in the planning image, identify an injection volume within the branch lumen, wherein the injection volume is to be injected with the therapeutic material; and
in the live procedural image, identify the injection volume within the branch lumen.

15. The system of claim 1, wherein the profile of the treatment device is a desired profile for a treatment device to be implanted or an actual profile of a treatment device already implanted.

16. The system of claim 1, wherein the first intraluminal device is a laser catheter device.

17. The system of claim 1, wherein the processor is further configured to identify the desired trajectory of the first intraluminal device such that a bend radius of the first intraluminal device is not smaller than a specified bend radius.

18. The system of claim 17, wherein the processor is further configured to identify the desired trajectory of the first intraluminal device such that a functional portion of the first intraluminal device approaches the desired puncture point within a desired range of angles.

19. A method for intraluminal fenestration within a body lumen, the method comprising:
with a processor in communication with a first intraluminal device comprising a flexible elongate member:
obtaining a planning image from a first imaging system, the planning image comprising the body lumen and a branch lumen extending from the body lumen;
in the planning image, identifying a profile of a treatment device;
in the planning image, identifying a centerline of the branch lumen, the centerline of the branch lumen extending from the branch lumen to a desired puncture point at a boundary of the profile of the treatment device;
in the planning image, identifying a desired trajectory of the first intraluminal device relative to the desired puncture point;
obtaining a live procedural image from a second imaging system, the live procedure image comprising the body lumen and the branch lumen extending from the body lumen; and
in the live procedural image, identifying the profile of the treatment device, the centerline of the branch lumen, the desired puncture point, the desired trajectory of the first intraluminal device, and an actual trajectory of the first intraluminal device.
